(19) United States
(12) Patent Application Publication
(10) Pub. No.: US 2022/0250003 A1
(43) Pub. Date: Aug. 11, 2022

(54) INHIBITOR REMOVAL SYSTEM FOR SENSOR DETECTION

(71) Applicant: TAESUNG ENVIRONMENTAL RESEARCH INSTITUTE CO., LTD., Ulsan (KR)

(72) Inventors: Gi Yeol YUN, Ulsan (KR); Seok Man KIM, Ulsan (KR)

(21) Appl. No.: 17/617,542

(22) PCT Filed: Dec. 6, 2019
(86) PCT No.: PCT/KR2019/017205

§ 371 Date: Dec. 8, 2021

(30) Foreign Application Priority Data
Dec. 4, 2019 (KR) 10-2019-0159687

(51) Int. Cl. B01D 53/75; B01D 46/44; B01D 45/16; B01D 46/00; B01D 46/42; B01D 50/20

(52) U.S. Cl. CPC B01D 53/75; B01D 46/44; B01D 50/20; B01D 46/0032; B01D 46/4263; B01D 45/16

(57) ABSTRACT

Provided in one embodiment of the present invention is an inhibitor removal system for sensor detection, comprising: a housing in which odorous gas flows in a pipe; a gas inlet part providing a passage through which the odorous gas flows into the housing; a pre-treatment part for removing odor detection inhibitors from the odorous gas flowing in the housing; a gas discharge part providing a passage through which the odorous gas is discharged from the housing; and a control unit for controlling the operation of a pre-treatment system in the pre-treatment part, wherein the pre-treatment part comprises a first pre-treatment part having a filtering device for filtering and separating out the odor detection inhibitors from the odorous gas; and/or a second pre-treatment part having an electrical dust collection device for collecting and separating out the odor detection inhibitors from the odorous gas.

### TECHNICAL FIELD

The present invention relates to an inhibitor removal system for sensor detection, and more particularly, to a pre-treatment device system for removing interfering elements such as dust, tar, moisture, and oil that interfere with the detection of a malodor sensor through a pre-treatment process of a malodor detection sensor.

### BACKGROUND ART

Malodor is defined as a smell in which irritative gaseous substances stimulate a person's sense of smell, thereby causing discomfort and disgust, and may be considered as one form of air pollution. In modern society, as the demand for a life of well-being is expanded, ways for alleviating discomfort and disgust caused by exposure to the malodor become a social issue. In particular, correct engineering technologies for prevention facilities or other facilities are being spotlighted to prevent malodors from chronic malodor emission facilities.

To this end, first, the fundamental cause of the malodor should be investigated, a solution suitable for the fundamental cause is derived, and thereafter, the design and operation plan of a malodor prevention facility should be presented. Thus, technologies for malodor analysis are necessarily required. In general, sampling, sensory evaluation, and device analysis are performed as processes for malodor analysis.

However, in the above-described process of analyzing a malodorous gas, inhibitors such as dust, tar, moisture, and oil are present in the malodorous gas in the atmosphere and are factors that hinder the accurate detection and analysis of an automatic malodor measurement sensor device or the like. Due to the above inhibitors, a measurement error may occur in detection data of a malodor sensor device, and further, a lifetime of a sensor device may be shortened. Accordingly, technologies for removing substances that inhibit malodor detection are required as a pre-treatment device of the malodor measurement sensor.

However, the related art document including Korean Patent Registration No. 10-1932678 (Title of invention: Determining apparatus for smell component and method thereof) merely suggests technologies for sequentially and precisely analyzing provided malodorous gas. That is, a technical problem of proposing a technical solution for removing malodorous gas inhibitors is still present as the pre-treatment technology for securing the reliability of measuring and analyzing the malodorous gas.

### RELATED ART DOCUMENT

(Patent Document 001) Korean Patent Registration No. 10-1932678 (registered on Dec. 19, 2018)

### DISCLOSURE

### Technical Problem

The present invention is directed to providing an inhibitor removal system for sensor detection including a filtering device or an air dilution-type electric dust collection device capable of separating malodor detection inhibitors in a pre-treatment process of a malodor measurement sensor.

### Technical Solution

One aspect of the present invention provides an inhibitor removal system for sensor detection including a housing part in which a malodorous gas flows along a pipe, a gas inlet that provides a passage through which the malodorous gas flows into the housing part, a pre-treatment unit that removes malodor detection inhibitors from the malodorous gas flowing in the housing part, a gas outlet that provides a passage through which the malodorous gas is discharged from the housing part, and a controller that adjusts an operation of a pre-treatment system of the pre-treatment unit, wherein the pre-treatment unit includes one or more of a first pre-treatment unit that is provided with a filtering device configured to filter and separate the malodor detection inhibitors from the malodorous gas, and a second pre-treatment unit that has an electric dust collection device configured to collect and separate the malodor detection inhibitors from the malodorous gas.

The first pre-treatment unit may further include a vortex separation unit that is provided with a hollow vortex space in which a vortex is formed in the malodorous gas and that separates the sinking malodor detection inhibitors from the vortex of the malodorous gas, a filter unit that transmits the malodorous gas and filters and separates the malodor detection inhibitors from the malodorous gas, and a condenser unit that cools and separates the malodor detection inhibitors from the malodorous gas.

The condenser unit may further include a coil pipe unit that is a flow pipe of the malodorous gas passing through the condenser unit and is provided with a flow section having a predetermined coil shape, a cooling casing unit that surrounds the coil pipe unit, one or more cooler units that are formed on one side of the cooling casing unit and reduce a temperature inside the coil pipe unit to a dew point or less, and a ventilator unit that discharges air from the cooler unit.

The filter unit may further include a first paper filter unit that filters the malodor detection inhibitors having a predetermined size or more, a second paper filter unit that filters the malodor detection inhibitors having a predetermined size or more and is located to be spaced apart from the first paper filter unit, and a control valve unit that controls opening/closing of a pipe so that the malodorous gas selectively flows into any one of the first paper filter unit and the second paper filter unit.

The second pre-treatment unit may further include an electric dust collector unit that collects the malodor detection inhibitors from the malodorous gas through electric discharge, and a heat exchanger unit that cools and separates the malodor detection inhibitors from the malodorous gas through heat exchange.

The electric dust collector unit may further include an electric discharge pipe unit that has a hollow internal space, extends to have a predetermined length, and collects the malodor detection inhibitors along an outer wall by electrically discharging in the internal space, and a voltage supply unit that provides a voltage to the internal space of the electric discharge pipe unit.

The heat exchanger unit may further include a heat exchange pipe unit that is a flow pipe for the malodorous gas passing through the heat exchanger unit and is provided with a flow section having a predetermined zigzag shape, a heat exchange casing unit that surrounds the heat exchange pipe unit and is formed so that cooling water flows in an internal space between the heat exchange casing unit and the heat exchange pipe unit, a cooling water inlet that provides a passage through which the cooling water flows into the heat exchange casing unit, and a cooling water outlet that provides a passage through which the cooling water is discharged from the heat exchange casing unit.

### Advantageous Effects

According to one aspect of the present invention, in a pre-treatment system through filtering and cooling, prior to malodor measurement, since substances such as dust, tar, and moisture are removed, a measurement error of a malodor detection sensor can be minimized, and a lifetime of a sensor device can be increased.

Further, in a pre-treatment system through air dilution-type electric dust collection and heat exchange prior to the malodor measurement, by pre-treating and cooling particulate pollutants such as dust and tar emitted at high temperatures, stable operation of the malodor detection sensor can be realized.

The effects of the present invention are not limited to the above effects and should be understood to include all effects that may be deduced from the detailed description of the present invention or the configuration of the present invention described in the appended claims.

### DESCRIPTION OF DRAWINGS

FIG. 1 is a front perspective view of an inhibitor removal system for sensor detection according to an embodiment of the present invention.

FIG. 2 is a front view of an exterior of the inhibitor removal system for sensor detection of FIG. 1.

FIG. 3 is a top perspective view of the inhibitor removal system for sensor detection of FIG. 1.

FIG. 4 is a front perspective view of an inhibitor removal system for sensor detection according to another embodiment of the present invention.

FIG. 5 is a side perspective view of the inhibitor removal system for sensor detection of FIG. 4.

### MODES OF THE INVENTION

Hereinafter, the present invention will be described with reference to the accompanying drawings. However, the present invention may be implemented in various different forms and thus is not limited to embodiments described herein. Further, in the drawings, parts irrelevant to the description are omitted in order to clearly describe the present invention, and throughout the specification, similar numerals reference numerals are assigned to similar parts.

Throughout the specification, when a first part is connected to a second part, this includes not only a case in which the first part is “directly connected” to the second part but also a case in which the first part is “indirectly connected” to the second part with a third part interposed therebetween. Further, when a part “includes” a component, this means that another component is not excluded but may be further included unless otherwise stated.

Hereinafter, embodiments of the present invention will be described in detail with reference to the accompanying drawings. Malodor detection inhibitors removed through a pre-treatment system of the present invention may be understood as particles such as dust and tar contained in malodorous gas, moisture, or oil.

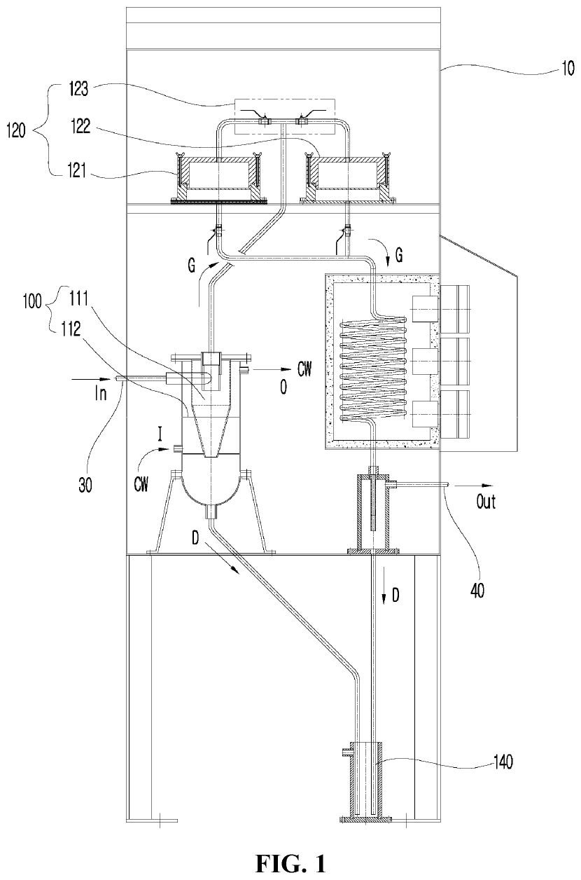

First, an inhibitor removal system for sensor detection according to a first embodiment of the present invention will be described with reference to FIGS. 1 to 3.

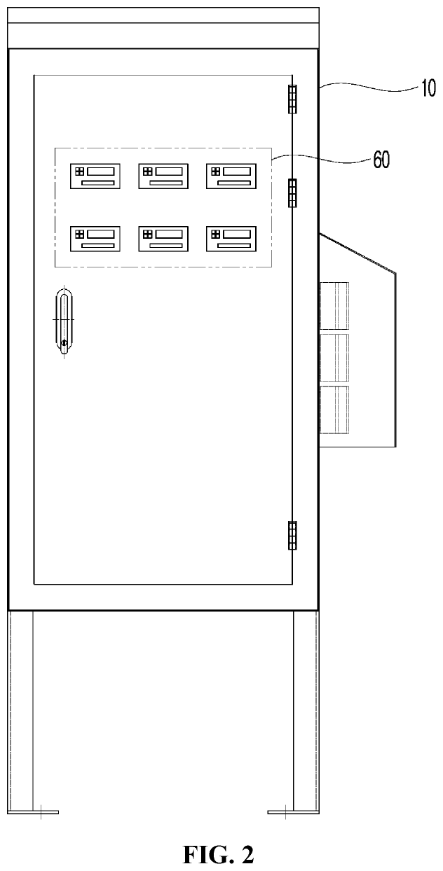

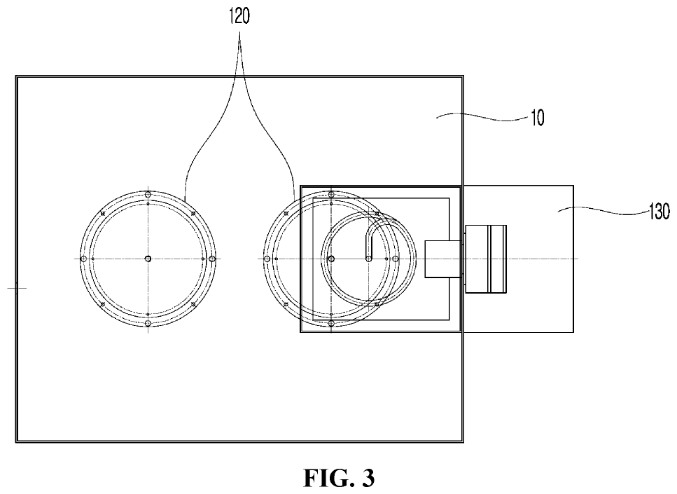

FIG. 1 is a front perspective view of an inhibitor removal system for sensor detection according to an embodiment of the present invention, FIG. 2 is a front view of an exterior of the inhibitor removal system for sensor detection of FIG. 1, and FIG. 3 is a top perspective view of the inhibitor removal system for sensor detection of FIG. 1.

As illustrated, the inhibitor removal system for sensor detection according to the first embodiment of the present invention includes, as components constituting a basic structure of the invention, a housing part 10, a gas inlet 30, a pre-treatment unit 20, a gas outlet 40, and a controller 50 (not illustrated), wherein the pre-treatment unit 20 is a first pre-treatment unit 100 having a filtering device for filtering and separating the malodor detection inhibitors from the malodorous gas.

In the housing part 10, the pre-treatment unit 20 is installed in a hollow space formed therein, and a pipe through which the introduced malodorous gas may flow via the pre-treatment unit 20 is installed. The pipe may be connected to a pump device that provides a suction force so that the malodorous gas may flow in a predetermined direction.

The gas inlet 30 is connected to one side of the housing part 10 and provides a passage through which the malodorous gas, which is a detection target of a malodor sensor, may flow into the housing part 10. The gas inlet 30 is located on the left side of the housing part 10 with reference to the accompanying drawings and is connected to the pipe inside the housing part 10 to provide the malodorous gas to the pre-treatment unit 20.

The gas outlet 40 is connected to the other side of the housing part 10, which is located opposite to the gas inlet 30, and provides a passage through which the malodorous gas, which is the detection target of the malodor sensor, may be discharged from the housing part 10. As illustrated, the gas outlet 40 is located on the right side of the housing part 10 and is connected to the pipe inside the housing part 10 to receive, from the pre-treatment unit 20, the malodorous gas from which the inhibitors are removed.

The gas outlet 40 is connected to a malodor measurement device including a malodor detection sensor and is installed to provide, to the malodor measurement device, the malodorous gas passing through the pre-treatment system according to the present invention.

The pre-treatment unit 20 is configured to perform a pre-treatment operation of removing the malodor detection inhibitors from the malodorous gas provided through the gas inlet 30. In this case, the controller 50 serves to control a pre-treatment operation system of the pre-treatment unit 20.

As described above, the pre-treatment unit 20 according to the embodiment of the present invention is a first pre-treatment unit 100 having the filtering device for filtering and separating the malodor detection inhibitors from the malodorous gas.

As illustrated, the first pre-treatment unit 100 according to the embodiment of the present invention may further include a vortex separation unit 110, a filter unit 120, a condenser unit 130, and an inhibitor collection unit 140, which may be sequentially arranged along the pipe according to a flow direction G of the malodorous gas.

The vortex separation unit 110 includes a vortex case 111 having a hollow vortex space into which the malodorous gas flows to form a vortex. The vortex case 111 is provided with an inlet on one side thereof into which the malodorous gas flows and is formed in a predetermined cylindrical shape to allow the malodorous gas to form the vortex therein.

As illustrated, the vortex case 111 according to the embodiment of the present invention may have a cylindrical shape in which a cross section thereof becomes narrower toward the lower side.

In this case, the malodor detection inhibitors such as various dusts and moisture contained in the malodorous gas are separated and sink due to the formed vortex. Thereafter, the malodorous gas is suctioned into the pipe formed above the vortex case 111 and flows upward, and the malodor detection inhibitors sink into the pipe formed below the vortex case 111 and flow downward by the gravity.

The vortex separation unit 110 according to the embodiment of the present invention may further include a vortex cover 112 having a housing shape that surrounds an outer surface of the vortex case 111 with a gap therebetween. The vortex cover 112 serves to allow the malodor detection inhibitors separated downward from the vortex case 111 to be delivered to a separate pipe without being dispersed.

In addition, the vortex cover 112 may be provided with an inlet and an outlet for cooling water on one side thereof and may have a cooling space formed therein in which the introduced cooling water and the vortex case 111 exchange heat with each other. That is, the cooling water is supplied to flow in the cooling space between the vortex cover 112 and the vortex case 111 and 111 so as to lower the temperature inside the vortex case 111 to a dew point or less.

Accordingly, in the vortex separation unit 110 of the present invention, a vortex operation on the malodorous gas is performed in the vortex space inside the vortex case 111 to separate the malodor detection inhibitors such as dust and tar, and at the same time, due to the cooling space outside the vortex case 111, the malodor detection inhibitors such as moisture generated from the malodorous gas may sink and be separated.

Through the vortex process described above, the malodor detection inhibitors such as dust, tar, and moisture discharged downward from the vortex separation unit 110 may be collected in the inhibitor collection unit 140 through the separate pipe.

The filter unit 120 has a secondary component for transmitting the malodorous gas from which the malodor detection inhibitors are primarily separated from the vortex separation unit 110 and filtering and separating the malodor detection inhibitors that are contained without being removed from the malodorous gas.

As illustrated, the filter unit 120 according to the embodiment of the present invention may further include a first paper filter unit 121, a second paper filter unit 122, and a control valve unit 123.

The first paper filter unit 121 and the second paper filter unit 122 are formed as a paper filter in the form of a mesh for filtering the malodor detection inhibitors such as dust and tar having a predetermined size or more from the malodorous gas and are installed to be spaced apart from each other so that the malodorous gas flows into an individual pipe. That is, a flow pipe for the malodorous gas introduced into the filter unit 120 is separated, and thus the malodorous gas selectively flows into the first paper filter unit 121 and the second paper filter unit 122.

The control valve unit 123, which is a valve component for controlling the opening and closing of the pipe connected to the filter unit 120, controls the pipe so that the malodorous gas selectively flows into one of the first paper filter unit 121 and the second paper filter unit 122.

According to the above structure, in the filter unit 120 according to the present invention, the control valve unit 123 allows the malodorous gas to selectively pass through any one of the first paper filter unit 120 or the second paper filter unit 122 so as to filter the malodor detection inhibitors such as dust and tar. In this case, the other paper filter unit through which the malodorous gas does not pass remains in a non-operating state, and thus a paper filter may be replaced or managed. That is, in the filter unit 120, since the first and second paper filter units 121 and 122 are selectively operated, a stable operation and a long-term lifetime of the filtering device may be achieved.

The condenser unit 130 is a component configured to allow the malodorous gas, from which the malodor detection inhibitors are secondarily separated by the filter unit 120, to pass therethrough and to tertiarily separate the malodor detection inhibitors from the malodorous gas by cooling the malodor detection inhibitors to the dew point or less and condensing the moisture. As illustrated, the condenser unit 130 according to the embodiment of the present invention may further include a coil pipe unit 131, a cooling casing unit 132, a cooler unit 133, and a ventilator unit 134.

The coil pipe unit 131 is a component configured to provide a flow pipe for the malodorous gas passing through the cooling casing unit 132 and having a predetermined coil-shaped flow section to maximize a pipe area to be cooled. The coil pipe unit 131 passing through the cooling casing unit 132 delivers the malodorous gas from a terminal thereof to the gas outlet 40.

The cooling casing unit 132 is a component provided with a hollow space therein and configured to surround the coil pipe unit 131 passing through the hollow space. The ventilator unit 134 is a component which is formed on one side of the cooling casing unit 132 and through which internal air is discharged and external air is introduced.

The cooler unit 133, which is one or more cooling device components formed on one side of the cooling casing unit 132, serves to lower, to the dew point or less, the temperature inside the coil pipe unit 131 through which the malodorous gas flows.

The cooler unit 133 according to the embodiment of the present invention is provided with a Peltier element. That is, the cooler unit 133 may be configured as one or more cooling devices using a Peltier phenomenon in which, after two different types of metal are joined, when a current passes therethrough, the joined part is cooled.

As illustrated in FIG. 1, the cooler unit 133 may have a plurality of cooling devices uniformly arranged to be spaced apart from each other in a height direction of the cooling casing unit 132 and thus perform extensive cooling in a direction of the coil pipe unit 131. When the temperature of the malodorous gas flowing inside the coil pipe unit 131 is lowered to the dew point or less, the inhibitors such as moisture are condensed and separated from the malodorous gas.

Through the condensing process described above, the malodor detection inhibitors such as dust, tar, and moisture discharged downward from the condenser unit 130 may be collected in the inhibitor collection unit 140 through the separate pipe.

Through the above structure, in the inhibitor removal system for sensor detection according to the first embodiment of the present invention, the introduced malodorous gas is subjected to a pre-treatment operation while sequentially passing through the vortex separation unit 110, the filter unit 120, and the condenser unit 130 of the first pre-treatment unit 100 and is then discharged. That is, using the vortex separation unit 110 and the filter unit 120, the dust and moisture contained in the high-temperature malodorous gas may be removed, and using the condenser unit 130, the temperature is lowered to the dew point or less, and thus the moisture may be removed.

Further, a display unit 60 formed outside the housing part 10 may disclose data information such as data on the temperature or content of the introduced malodorous gas, the temperature of the condenser unit 130, and the temperature after the cooling. Further, by inputting a control value of the controller 50 through the display unit 60, the inhibitor removal system for sensor detection according to the present invention may be controlled.

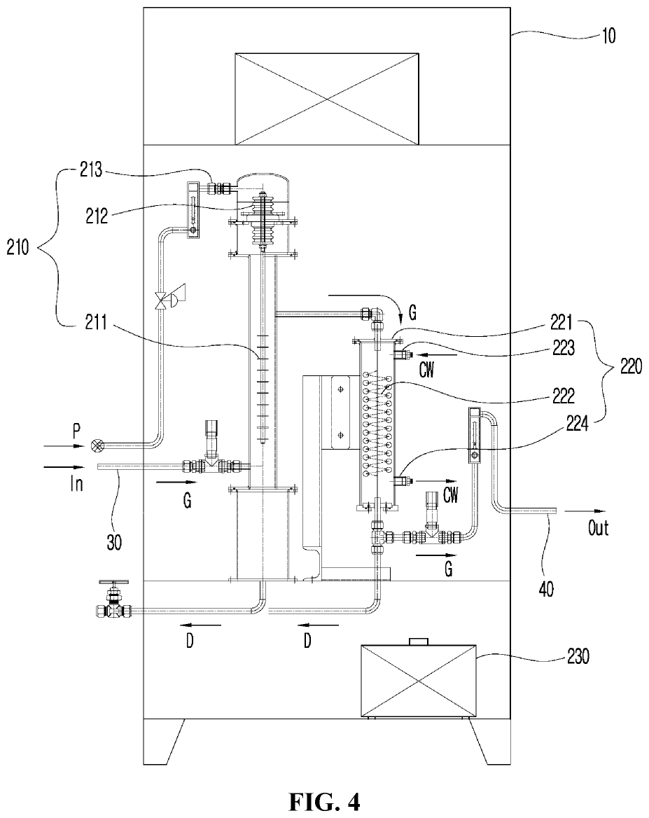

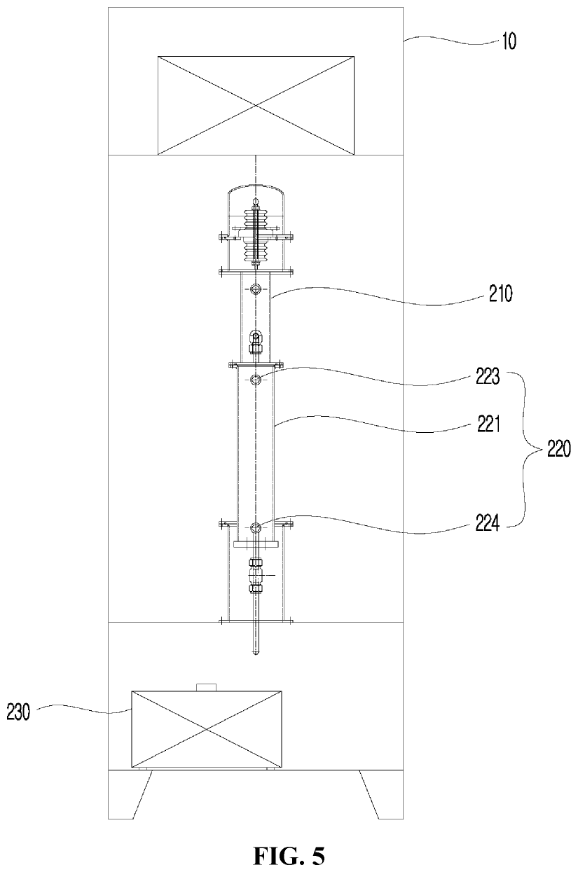

Hereinafter, an inhibitor removal system for sensor detection according to a second embodiment of the present invention will be described with reference to FIGS. 4 to 5.

FIG. 4 is a front perspective view of an inhibitor removal system for sensor detection according to the second embodiment of the present invention, and FIG. 5 is a side perspective view of the inhibitor removal system for sensor detection of FIG. 4.

As illustrated, the inhibitor removal system for sensor detection according to the second embodiment of the present invention includes, as components constituting a basic structure of the invention, a housing part 10, a gas inlet 30, a pre-treatment unit 20, a gas outlet 40, and a controller 50 (not illustrated), wherein the pre-treatment unit 20 is a second pre-treatment unit 200 having a filtering device for filtering and separating the malodor detection inhibitors from the malodorous gas.

The housing part 10, the gas inlet 30, and the gas outlet 40 according to the second embodiment of the present invention may be understood as being the same as the housing part 10, the gas inlet 30, and the gas outlet 40 according to the first embodiment of the present invention, which are described above. The detailed description therefor will be omitted.

As illustrated, the second pre-treatment unit 200 according to the embodiment of the present invention may further include an electric dust collector unit 210, a heat exchanger unit 220, and a cooling water supply unit 230 which may be sequentially arranged along the pipe according to the flow direction G of the malodorous gas.

The electric dust collector unit 210 is a component configured to receive the malodorous gas from the gas inlet 30 and remove the malodor detection inhibitors from the malodorous gas in a predetermined pipe housing, in which the malodorous gas moves upward, using air dilution-type electric dust collection.

As illustrated, the electric dust collector unit 201 according to the embodiment of the present invention may further include an electric discharge pipe unit 211, a voltage supply unit 212, and a purge pressure unit 213. In addition, although not illustrated in the drawings, the electric dust collector unit 210 may further include a separate voltage increasing device that raises and transmits a voltage provided to the electric dust collector unit 210.

The electric discharge pipe unit 211 is formed to extend to have a predetermined length in a height direction inside the pipe housing constituting the electric dust collector unit 210. In this case, an internal space receives a high voltage, and thus discharge occurs, and dust is collected on an outer wall.

In the electric discharge pipe unit 211 according to the embodiment of the present invention, corona discharge as a form of discharge in a gas may be applied in which, when a high voltage is applied between two electrodes, only a part having a strong electric field emits light before a spark is emitted, and thus conductivity is achieved.

The voltage supply unit 212 is a component configured to receive a high voltage from the separate voltage increasing device and provide a voltage so that electric discharge may occur in the internal space of the electric discharge pipe unit 211. The purge pressure unit 213 serves to provide a pneumatic pressure downward from an upper end of the pipe housing in order to prevent the malodorous gas from flowing upward in reverse.

In description of a dust collection operation of the electric dust collector unit 210 of the present invention through the above structure, the malodorous gas introduced from the gas inlet on one lower side of the pipe housing flows upward along the inside of the pipe housing. In this case, the high voltage increased by the separate voltage increasing device is provided to the voltage supply unit 212, and this voltage is transmitted to the internal space of the electric discharge pipe unit 211.

The corona discharge occurs in the internal space of the electric discharge pipe unit 211 due to collision between electrons according to the movement of the electrons in the gas. Accordingly, dust, tar, and the like are collected and separated from the flowing malodorous gas on an outer wall surface of the electric discharge pipe unit 211. In this case, the purge pressure unit 213 provides a pneumatic pressure downward from the upper end of the pipe housing to prevent the malodorous gas from flowing upward in reverse so as to discharge the malodorous gas to the flow pipe.

Thereafter, unlike the malodorous gas, the malodor detection inhibitors, which are separated from the dust, sink down to the bottom of the pipe housing. Through the electric dust collection process described above, the malodor detection inhibitors such as dust and tar discharged downward from the electric dust collector unit 210 may be collected in the inhibitor collection unit 240 (not illustrated) through the separate pipe.

The heat exchanger unit 220 is a component configured to receive the malodorous gas from which the malodor detection inhibitors are primarily separated by the electric dust collector unit 210 and to cool the malodor detection inhibitors through heat exchange so as to secondarily separate the moisture from the malodorous gas.

The cooling water supply unit 230 serves to supply the cooling water used for heat exchange of the heat exchanger unit 220.

As illustrated, the heat exchanger unit 220 according to the embodiment of the present invention may further include a heat exchange pipe unit 222, a heat exchange casing unit 221, a cooling water inlet 223, and a cooling water outlet 224.

The heat exchange casing unit 221, which is a casing surrounding the heat exchange pipe unit 222, is formed so that the cooling water flows in an internal space between the heat exchange casing unit 221 and the heat exchange pipe unit 222. The cooling water inlet 223 and the cooling water outlet 224 provide a passage having one end connected to the cooling water supply unit 230 and the other end through which the cooling water flows into or is discharged from the heat exchange casing unit 221.

The heat exchange pipe unit 222 is a component corresponding to the flow pipe of the malodorous gas passing downward through the heat exchange casing unit 221. The heat exchange pipe unit 222 according to the embodiment of the present invention may provide a predetermined zigzag-shaped or coil-shaped flow section in order to maximize a contact area for heat exchange with the cooling water. The heat exchange pipe unit 222 lowers the temperature of the internal space to the dew point or less through the heat exchange with the cooling water from the outside and thus separates the malodor detection inhibitors from the malodorous gas.

Through the heat exchange process described above, the malodor detection inhibitors such as dust, tar, and moisture discharged downward from the heat exchanger unit 220 may be collected in the inhibitor collection unit 240 through the separate pipe.

Through the above structure, in the inhibitor removal system for sensor detection according to the second embodiment of the present invention, the introduced malodorous gas is subjected to a pre-treatment operation while sequentially passing through the electric dust collector unit 210 and the heat exchanger unit 220 of the second pre-treatment unit 200 and is then discharged. That is, the electric dust collector unit 210 may perform electric dust collection to remove the dust and moisture contained in the high-temperature malodorous gas, and using the heat exchanger unit 220, the temperature is lowered to the dew point or less, and thus the moisture may be removed.

Further, the display unit 60 (not illustrated) formed outside the housing part 10 may disclose data information such as data on the temperature or content of the introduced malodorous gas, the temperature of the heat exchanger unit 220, and the temperature after the cooling. Further, by inputting a control value of the controller 50 through the display unit 60, the inhibitor removal system for sensor detection according to the present invention may be controlled.

Meanwhile, the controller 50 according to the embodiment of the present invention includes a sensor that detects data on the temperature, dust, the moisture content, the oil content, and the like of the malodorous gas introduced into the housing part, and the controller 50 controls, according to a preset condition based on the detected data, the pre-treatment system to be operated selectively by any one of the first pre-treatment unit 100 or the second pre-treatment unit 200 or to be operated sequentially by the first pre-treatment unit 100 and the second pre-treatment unit 200.

According to the above technical features, the inhibitor removal system for sensor detection according to the present invention is a pre-treatment system through the filtering and the air dilution-type electric dust collection prior to the malodor measurement, wherein as the malodorous gas from which substances such as dust, tar, and moisture are removed is provided, a measurement error of the malodor detection sensor can be minimized, a lifetime of a sensor device can be increased, and the malodor detection sensor can be stably operated.

The above description of the present invention is merely illustrative, and those skilled in the art to which the present invention pertains can understand that the present invention can be easily modified in other specific forms without changing the technical spirit or essential features of the present invention. Therefore, it should be understood that the embodiments described above are illustrative but not limiting in all aspects. For example, components described as a single type may be implemented in a distributed manner, and likewise, components described in a distributed manner may also be implemented in a coupled form.

The scope of the present invention is indicated by the appended claims, and all changes or modifications derived from the meaning and scope of the appended claims and equivalent concepts thereof should be construed as being included in the scope of the present invention.

### DESCRIPTION OF REFERENCE NUMERALS

10: Housing part
20: Pre-treatment unit
30: Gas inlet
40: Gas outlet
50: Controller
60: Display unit
100: First pre-treatment unit
110: Vortex separation unit
111: Vortex case
112: Vortex cover
120: Filter unit
121: First paper filter unit
122: Second paper filter unit
123: Control valve unit
130: Condenser unit
131: Coil pipe unit
132: Cooling casing unit
133: Cooler unit
134: Ventilator unit
140: Inhibitor collection unit
200: Second pre-treatment unit
210: Electric dust collector unit
211: Electric discharge pipe unit
212: Voltage supply unit
213: Purge pressure unit
220: Heat exchanger unit
221: Heat exchange casing unit
222: Heat exchange pipe unit
223: Cooling water inlet
224: Cooling water outlet
230: Cooling water supply unit
240: Inhibitor collection unit

What is claimed is:

1. An inhibitor removal system for sensor detection, comprising:
a housing part in which a malodorous gas flows along a pipe;
a gas inlet that provides a passage through which the malodorous gas flows into the housing part;
a pre-treatment unit that removes malodor detection inhibitors from the malodorous gas flowing in the housing part;
a gas outlet that provides a passage through which the malodorous gas is discharged from the housing part; and
a controller that adjusts an operation of a pre-treatment system of the pre-treatment unit,
wherein the pre-treatment unit includes one or more of:
a first pre-treatment unit that is provided with a filtering device configured to filter and separate the malodor detection inhibitors from the malodorous gas; and
a second pre-treatment unit that has an electric dust collection device configured to collect and separate the malodor detection inhibitors from the malodorous gas.

2. The inhibitor removal system of claim 1, wherein the first pre-treatment unit further includes:
a vortex separation unit that is provided with a hollow vortex space in which a vortex is formed in the malodorous gas and that separates the sinking malodor detection inhibitors from the vortex of the malodorous gas;
a filter unit that transmits the malodorous gas and filters and separates the malodor detection inhibitors from the malodorous gas; and
a condenser unit that cools and separates the malodor detection inhibitors from the malodorous gas.

3. The inhibitor removal system of claim 2, wherein the condenser unit further includes:
a coil pipe unit that is a flow pipe of the malodorous gas passing through the condenser unit and is provided with a flow section having a predetermined coil shape;
a cooling casing unit that surrounds the coil pipe unit;
one or more cooler units that are formed on one side of the cooling casing unit and reduce a temperature inside the coil pipe unit to a dew point or less; and
a ventilator unit that discharges air from the cooler unit.

4. The inhibitor removal system of claim 2, wherein the filter unit further includes:
a first paper filter unit that filters the malodor detection inhibitors having a predetermined size or more;
a second paper filter unit that filters the malodor detection inhibitors having a predetermined size or more and is located to be spaced apart from the first paper filter unit; and
a control valve unit that controls opening/closing of a pipe so that the malodorous gas selectively flows into any one of the first paper filter unit and the second paper filter unit.

5. The inhibitor removal system of claim 1, wherein the second pre-treatment unit further includes:
an electric dust collector unit that collects the malodor detection inhibitors from the malodorous gas through electric discharge; and
a heat exchanger unit that cools and separates the malodor detection inhibitors from the malodorous gas through heat exchange.

6. The inhibitor removal system of claim 5, wherein the electric dust collector unit further includes:
an electric discharge pipe unit that has a hollow internal space, extends to have a predetermined length, and collects the malodor detection inhibitors along an outer wall by electrically discharging in the internal space; and
a voltage supply unit that provides a voltage to the internal space of the electric discharge pipe unit.

7. The inhibitor removal system of claim 5, wherein the heat exchanger unit further includes:
a heat exchange pipe unit that is a flow pipe for the malodorous gas passing through the heat exchanger unit and is provided with a flow section having a predetermined zigzag shape;
a heat exchange casing unit that surrounds the heat exchange pipe unit and is formed so that cooling water flows in an internal space between the heat exchange casing unit and the heat exchange pipe unit;
a cooling water inlet that provides a passage through which the cooling water flows into the heat exchange casing unit; and
a cooling water outlet that provides a passage through which the cooling water is discharged from the heat exchange casing unit.
